(19) United States
(12) Patent Application Publication
(10) Pub. No.: US 2023/0273048 A1
(43) Pub. Date: Aug. 31, 2023

(54) INFORMATION PROCESSING APPARATUS, INFORMATION PROCESSING SYSTEM, INFORMATION PROCESSING METHOD, AND PROGRAM

(71) Applicant: SEIKO EPSON CORPORATION, Tokyo (JP)

(72) Inventor: Kohei ITO, Shiojiri-shi (JP)

(73) Assignee: SEIKO EPSON CORPORATION, Tokyo (JP)

(21) Appl. No.: 18/174,060

(22) Filed: Feb. 24, 2023

(30) Foreign Application Priority Data
Feb. 25, 2022 (JP) 2022-027916

(51) Int. Cl. G01C 22/00; A61B 5/103; A61B 5/00

(52) U.S. Cl. CPC G01C 22/006; A61B 5/1038; A61B 5/6807; A61B 2503/12

(57) ABSTRACT

An information processing apparatus acquires information indicating how the feet of a user wearing shoes move based on data acquired by an electronic device attached to the shoes and determines whether the user is tired based on the acquired information.

The present application is based on, and claims priority from JP Application Serial Number 2022-027916, filed Feb. 25, 2022, the disclosure of which is hereby incorporated by reference herein in its entirety.

### BACKGROUND

### 1. Technical Field

This disclosure relates to an information processing apparatus, an information processing system, an information processing method, and a program.

### 2. Related Art

Technologies for supporting people's lives have been researched and developed.

In this regard, an information processing apparatus for acquiring information indicating the number of impacts given while a user is exercising from an electronic device attached to the shoes that the user is wearing and determining whether the user is tired based on the acquired information is known (see JP-A-6-054837).

However, when the number of impacts indicated by the acquired information is less than a predetermined threshold, the information processing apparatus described in JP-A-6-054837 may determine that the user is tired, regardless of whether the user is actually tired.

### SUMMARY

An aspect of this disclosure to solve the problem described above is an information processing apparatus configured to acquire information indicating how a foot of a user wearing a shoe moves based on data acquired by an electronic device attached to the shoe and determine whether the user is tired based on the acquired information.

In addition, an aspect of this disclosure is an information processing system including the information processing apparatus described above and the electronic device.

In addition, an aspect of this disclosure is an information processing method for an information processing apparatus, the information processing method including receiving data acquired by an electronic device attached to a shoe, acquiring information indicating how a foot of a user wearing the shoe moves based on the received data, and determining whether the user is tired based on the acquired information.

In addition, an aspect of this disclosure is a non-transitory computer-readable storage medium storing a program, the program being configured to cause a computer of an information processing apparatus to receive data acquired by an electronic device attached to a shoe, acquire information indicating how a foot of a user wearing the shoe moves based on the received data, and determine whether the user is tired based on the acquired information.

In addition, an aspect of this disclosure is an information processing apparatus configured to acquire information indicating how a foot of a user wearing a shoe moves based on data acquired by an electronic device attached to the shoe and determine whether the user is injured based on the acquired information.

### BRIEF DESCRIPTION OF THE DRAWINGS

FIG. 1 is a diagram illustrating an example of a configuration of an information processing system 1.

FIG. 2 is a diagram illustrating an example of a hardware configuration of an electronic device 10.

FIG. 3 is a diagram illustrating an example of a hardware configuration of an information processing apparatus 20.

FIG. 4 is a diagram illustrating an example of a hardware configuration of a mobile terminal 30.

FIG. 5 is a diagram illustrating an example of functional configurations of the electronic device 10 and the information processing apparatus 20.

FIG. 6 is a diagram illustrating an example of a process flow for the information processing apparatus 20 to determine whether a user U is tired.

FIG. 7 is a diagram illustrating an example of a process flow in which the information processing apparatus 20 acquires detection data used to generate determination criterion information.

FIG. 8 is a diagram illustrating an example of a process flow in which the information processing apparatus 20 generates determination criterion information based on detection data acquired in the processing of the flowchart illustrated in FIG. 7.

### DESCRIPTION OF EXEMPLARY EMBODIMENTS

### Embodiments

Exemplary embodiments of this disclosure will be described below with reference to the drawings.

### Overview of Information Processing System

First, an overview of an information processing system according to an embodiment will be described.

The information processing system according to an embodiment includes an electronic device and an information processing apparatus. The electronic device is attached to shoes. The information processing apparatus acquires information indicating how the user's feet wearing the shoes move based on data acquired by the electronic device and determines whether the user is tired based on the acquired information. This allows the information processing system to determine whether the user is tired based on how the user's feet move.

A configuration of the information processing system and processing of the information processing system according to an embodiment will be described in detail below.

### Configuration of Information Processing System

Hereinafter, a configuration of the information processing system according to an embodiment will be described exemplifying an information processing system 1.

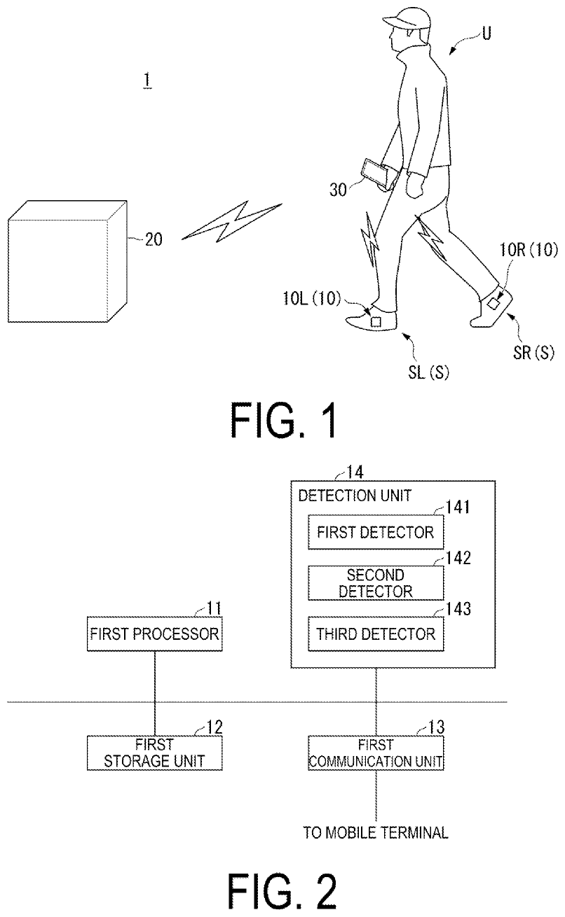

FIG. 1 is a diagram illustrating an example of a configuration of the information processing system 1. As an example, a case in which a user of the information processing system 1 is a user U illustrated in FIG. 1 will be described below.

The information processing system 1 determines whether the user U is tired in the period in which the user U is walking. In addition, the information processing system 1 performs processing in accordance with the result of the determination of whether the user U is tired. Although the processing may be, for example, processing of outputting meal menu information indicating a menu for a meal in accordance with the result of determination of whether the user U is tired, it is not limited thereto. Here, although the period in which the user U is walking is a period specified by the user U, for example, a period in which the user U is going to work, a period in which the user U is going home, a period in which the user U is taking a walk, a period in which the user U is racewalking, a period in which the user U is working, or a period in which the user U is exercising, it is not limited thereto. The period in which the user U is exercising is, for example, a period in which the user U is running a marathon. A period in which the user U is walking will be referred to as a “measurement period” below for the sake of convenience of description.

The information processing system 1 includes, for example, an electronic device 10, and an information processing apparatus 20, and a mobile terminal 30 as illustrated in FIG. 1. Further, in the information processing system 1, some or all of the electronic device 10, the information processing apparatus 20, and the mobile terminal 30 may be integrated. In addition, the information processing system 1 may be configured not to include the mobile terminal 30.

The electronic device 10 is attached to shoes that the user U is wearing when the information processing system 1 is used. The electronic device 10 includes a left-foot electronic device 10L attached to the shoe that the user U wears on his or her left foot, and a right-foot electronic device 10R attached to the shoe that the user U wears on his or her right foot. For the sake of convenience of explanation, the shoes that the user U wears when using the information processing system 1 will be referred to as “first shoes S” below. In addition, hereinafter, for the sake of convenience of explanation, the shoe that the user U wears on his or her left foot will be referred to as a “left foot shoe SL”, and the shoe that the user U wears on his or her right foot will be referred to as a “right foot shoe SR”. Here, the first shoes S may be shoes used for sports such as running shoes, and may be leather shoes, or other types of shoes.

Also, the electronic device 10 may be separate from the first shoes S, or may be integrated with the first shoes S. When the first shoes S are integrated, the left-foot electronic device 10L is integrated with the left foot shoe SL. In addition, in this case, the right-foot electronic device 10R is integrated with the right foot shoe SR. In the following, a case in which the electronic device 10 is separate bodies from the first shoes S will be described as an example. In this case, the left-foot electronic device 10L is attached to the left foot shoe SL at the outer side of the left foot shoe SL. In addition, in this case, the right-foot electronic device 10R is attached to the right foot shoe SR at the outer side of the right foot shoe SR. These attachments may be achieved in a method using a restraint such as a belt, or may be achieved in any other method using other instruments, jigs, equipment, and the like.

The left-foot electronic device 10L acquires one or more pieces of data indicating how the left-foot electronic device 10L moves. In the following, as an example, a case in which the left-foot electronic device 10L acquires three kinds of data including acceleration data indicating an acceleration of the left-foot electronic device 10L, angular velocity data indicating an angular velocity of the left-foot electronic device 10L, and position data indicating a position of the left-foot electronic device 10L will be described. In this case, the left-foot electronic device 10L includes an acceleration sensor that detects an acceleration, an angular velocity sensor that detects an angular velocity, and a position data receiver that receives position data indicating a position. Here, the angular velocity sensor is a sensor capable of detecting an angular velocity of a gyro sensor, for example. In addition, the position data receiver is a receiving apparatus that receives data indicating the position measured by a Global Navigation Satellite System (GNSS) as position data indicating a position of the left-foot electronic device 10L, and is, for example, a Global Positioning System (GPS) receiver. Thus, the position data received by the position data receiver includes speed data indicating a speed of the left-foot electronic device 10L and date-and-time data indicating the date and time on which the position indicated by the position data is measured.

The left-foot electronic device 10L acquires acceleration data indicating an acceleration detected by the acceleration sensor from the acceleration sensor as user left-foot acceleration data indicating the acceleration of the left foot of the user U. In addition, the left-foot electronic device 10L acquires angular velocity data indicating an angular velocity detected by the angular velocity sensor from the angular velocity sensor as user left-foot angular velocity data indicating the angular velocity of the left foot of the user U. In addition, the left-foot electronic device 10L acquires position data received by the position data receiver from the position data receiver as user left-foot position data indicating the position of the left foot of the user U. Further, the left-foot electronic device 10L may be configured to acquire one or more pieces of other data indicating how the left-foot electronic device 10L moves instead of some or all of the user left-foot acceleration data, the user left-foot angular velocity data, and the user left-foot position data, or in addition to all of the user left-foot acceleration data, the user left-foot angular velocity data, and the user left-foot position data. In this case, the left-foot electronic device 10L includes one or more sensors that detect each of one or more pieces of the other data. Furthermore, the left-foot electronic device 10L may be configured to acquire some of the user left-foot acceleration data, the user left-foot angular velocity data, and the user left-foot position data. Here, the left-foot electronic device 10L may have a configuration without an acceleration sensor when user left-foot acceleration data is not acquired. In addition, the left-foot electronic device 10L may have a configuration without an angular velocity sensor when user left-foot angular velocity data is not acquired. In addition, the left-foot electronic device 10L may have a configuration without a position data receiver when user left-foot position data is not acquired. Hereinafter, for the sake of convenience of explanation, user left-foot acceleration data, user left-foot angular velocity data, and user left-foot position data will be collectively referred to as detection data as long as there is no need to distinguish those kinds of data.

In addition, the left-foot electronic device 10L acquires detection data each time a predetermined sampling period elapses. Although the predetermined sampling period is, for example, a few milliseconds, it is not limited to thereto. The left-foot electronic device 10L transmits the acquired detection data to the information processing apparatus 20 via the mobile terminal 30 each time detection data is acquired.

The left-foot electronic device 10L transmits and/or receives various kinds of data to and from the mobile terminal 30 in wireless communication based on a predetermined first standard. The first standard may be, for example, the standard of Bluetooth (trade name), the standard of Wi-Fi (trade name), or another standard for wireless communication.

The right-foot electronic device 10R may have a similar configuration to that of the left-foot electronic device 10L, and may have a different configuration from that as long as the functions of the information processing system 1 described in this embodiment are not impaired. A case in which the right-foot electronic device 10R has a similar configuration to that of the left-foot electronic device 10L will be described below as an example. In this case, the right-foot electronic device 10R acquires detection data about the right foot of the user U each time the predetermined sampling period elapses. In addition, the right-foot electronic device 10R transmits the acquired detection data to the information processing apparatus 20 via the mobile terminal 30 each time detection data is acquired.

Each of the left-foot electronic device 10L and the right-foot electronic device 10R may be controlled by one processor mounted on either of the left-foot electronic device 10L and the right-foot electronic device 10R, or may be controlled by processors installed independently of each other. In the following, a case in which the left-foot electronic device 10L and the right-foot electronic device 10R are controlled by processors installed independently of each other will be described as an example. Furthermore, timings at which the left-foot electronic device 10L and the right-foot electronic device 10R acquire and transmit detection data may or may not be synchronized with each other. In the following, a case in which timings at which the left-foot electronic device 10L and the right-foot electronic device 10R acquire and transmit detection data are synchronized with each other will be described as an example. A method for achieving the configuration may be a known method, or may be a method to be developed in the future.

As described above, in this embodiment, the left-foot electronic device 10L and the right-foot electronic device 10R have the same configuration. For this reason, for the sake of convenience of description, the configurations of the left-foot electronic device 10L and the right-foot electronic device 10R will be collectively described as a configuration of an electronic device 10 below as long as the configurations need not be distinguished from each other. Furthermore, for the sake of convenience of description, the operations and processing of the left-foot electronic device 10L and the right-foot electronic device 10R will be collectively described as an operation and processing of the electronic device 10 below as long as the operations and processing need not be distinguished from each other. Thus, in the following, detection data acquired by the electronic device 10 means the two kinds of detection data including detection data acquired by the left-foot electronic device 10L and detection data acquired by the right-foot electronic device 10R.

Further, the information processing system 1 may have a configuration in which either of the left-foot electronic device 10L and the right-foot electronic device 10R is included as the electronic device 10. In this case, the information processing system 1 uses, for example, either of detection data for the left foot of the user U and detection data for the right foot of the user U as detection data for both feet of the user U. In addition, in this case, the information processing system 1, for example, attaches the electronic device 10 to the left foot shoe SL when detection data for the left foot of the user U is to be acquired, and attaches the electronic device 10 to the right foot shoe SR when detection data for the right foot of the user U is to be acquired.

The information processing apparatus 20 may be any information processing apparatus as long as the information processing apparatus can function as a server. Although the information processing apparatus 20 is, for example, a desktop personal computer (PC) or a workstation, it is not limited thereto.

The information processing apparatus 20 receives, from the electronic device 10, the detection data acquired by the electronic device 10 via the mobile terminal 30 in the measurement period described above. More specifically, the measurement period is a period in which the information processing system 1 acquires detection data using the electronic device 10, and is, for example, a period from the timing at which the information processing apparatus 20 receives, from the user, an operation to start acquisition of detection data via the mobile terminal 30 to the timing at which the information processing apparatus 20 receives, from the user, an operation to end the acquisition of the detection data via the mobile terminal 30.

The information processing apparatus 20 acquires one or more types of first information indicating how the feet of the user U move based on the detection data received in the measurement period. Here, the one or more types of first information include, for example, user stride-length information indicating the stride length of the user U, and user pitch information indicating the pitch of the user U. Further, the information indicating the number of impacts imparted to the first shoes S while the user U is walking is not suitable as the one or more kinds of first information. This is because the impacts are not attributable only to how the feet of the user U move. For this reason, in this embodiment, the information indicating the number of impacts is not the information indicating how the feet of the user U move. In the following, a case in which the one or more kinds of first information is user stride-length information will be described as an example. In this case, the information processing apparatus 20 calculates the stride length of the left foot of the user U and the stride length of the right foot of the user U based on the detection data received in the measurement period. Here, the stride length of the left foot of the user U may be calculated based on user left-foot acceleration data, based on a combination of user left-foot acceleration data and user left-foot angular velocity data, based on a combination of user left-foot acceleration data, user left-foot angular velocity data, and user left-foot position data, based on a combination of user left-foot angular velocity data and user left-foot position data, or based on user left-foot position data. In addition, the stride length of the right foot of the user U may be calculated based on user right-foot acceleration data, based on a combination of user right-foot acceleration data and user right-foot angular velocity data, based on a combination of user right-foot acceleration data, user right-foot angular velocity data, and user right-foot position data, based on a combination of user right-foot angular velocity data and user right-foot position data, or based on user right-foot position data. In addition, the information processing apparatus 20 calculates the average value of the calculated stride length of the left foot of the user U and the calculated stride length of the right foot of the user U as a stride length of the user U. In this manner, the information processing apparatus 20 acquires the user stride-length information indicating the stride length of the user U. Further, the information processing apparatus 20 may calculate either of the stride length of the left foot of the user U and the stride length of the right foot of the user U as a stride length of the user U. Furthermore, a method for calculating each of the stride length of the left foot of the user U and the stride length of the right foot of the user U based on some or all of the detection data may be a known method, or a method to be developed in the future.

Furthermore, the information processing apparatus 20 acquires walking-related information about walking of the user U wearing the first shoes S based on the detection data received in the measurement period. The walking-related information is information including one or more kinds of information about walking of the user U. In the following, as an example, a case in which walking-related information includes three kinds of information such as road surface slope information indicating the slope of the road surface on which the user U is walking, user walking speed information indicating the speed of the walking user U, and user walking time slot information indicating the time slot in which the user is walking will be described. The information processing apparatus 20 calculates, for example, the slope of the road surface on which the user U is walking at each time in the measurement period based on at least one of user left-foot angular velocity data included in the detection data or user right-foot angular velocity data included in the detection data. A method of calculating the slope of the road surface on which the user U is walking at each time in the measurement period based on at least one of the user left-foot angular velocity data or the user right-foot angular velocity data may be a known method, or a method to be developed in the future. Furthermore, the information processing apparatus 20 calculates the speed of the user U at each time in the measurement period, for example, based on the detection data. Here, a speed of the user U may be calculated based on user left-foot acceleration data, based on a combination of user left-foot acceleration data and user left-foot angular velocity data, based on a combination of user left-foot acceleration data, user left-foot angular velocity data, and user left-foot position data, or based on a combination of user left-foot angular velocity data and user left-foot position data. Furthermore, a method of calculating a speed of the user U based on some or all of detection data may be a known method or a method to be developed in the future. In addition, the information processing apparatus 20 may identify the average value of speeds indicated by speed data included in each of left foot position data and right foot position data as a speed of the user U. Furthermore, the information processing apparatus 20 identifies, for example, the time slot included in the measurement period based on a timestamp of each piece of data included in the detection data. Further, the information processing apparatus 20 may identify, for example, the time slot included in the measurement period based on the date and time indicated by date-and-time data included in each of the left foot position data and the right foot position data. As described above, the information processing apparatus 20 acquires the walking-related information based on the detection data received in the measurement period. Further, the walking-related information may include other kinds of information about walking of the user U wearing the first shoes S, instead of some or all of the road surface slope information, the user walking speed information, and the user walking time slot information, or in addition to all of the road surface slope information, the user walking speed information, and the user walking time slot information. In this case, the information processing apparatus 20 acquires the other information based on the detection data received in the measurement period. Furthermore, the walking-related information may include some of the road surface slope information, the user walking speed information, and the user walking time slot information.

Here, determination criterion information is stored in advance in the information processing apparatus 20. The determination criterion information is information used to determine whether the user U is tired. The determination criterion information is information including threshold information indicating a threshold for a stride length of the user U. In the following, as an example, a case in which the determination criterion information is information in which the walking-related information is associated with threshold information for each of a plurality of different pieces of walking-related information will be described. Here, that certain walking-related information A is different from walking-related information B that is different from the walking-related information A means that a combination of the values indicated by each of one or more kinds of information included in the walking-related information A does not match a combination of values indicated by each of one or more kinds of information included in the walking-related information B. Furthermore, some or all of the threshold information associated with each piece of the plurality of walking-related information included in the determination criterion information may indicate the same threshold, or different thresholds. Furthermore, the determination criterion information may be information in a table format, or information stored as a weight between nodes in a machine learning model, or information in another format. In addition, the determination criterion information may be stored in the information processing apparatus 20 in advance by the user U, or may be generated by the information processing apparatus 20 based on the detection data acquired from the electronic device 10.

In addition, certain walking-related information can be said to be information indicating a situation in which the user U is walking. Furthermore, a stride length of the user U tends to be shorter when the user U is climbing a slope surface, for example, compared to that when the user U is walking on a flat surface. Also, a stride length of the user U tends to be longer when the user U is going down a slope surface, for example, compared to that when the user U is walking on a flat surface. Meanwhile, a stride length of the user U tends to be shorter when the user U is tired, for example, compared to that when the user U is not tired. In addition, a stride length of the user U tends to be longer when the user U is not tired, for example, compared to that when the user U is tired. For those reasons, as a threshold indicated by threshold information associated with certain walking-related information, the stride length of the user U when the user U is not tired in a situation indicated by the walking-related information may be employed in the determination criterion information. As a result, the information processing apparatus 20 can prevent the user U from being determined as being tired even though the user U is not tired and as not being tired even though the user is tired with reference to the difference between the stride length when the user U who is not tired is climbing a slope surface and the stride length when the user U who is not tired is walking on a flat surface. In other words, the information processing apparatus 20 can prevent the user U from being incorrectly determined as being tired even though the user U is not tired based on the difference in stride length of the user U made according to a situation where the user U is walking. In addition, the information processing apparatus 20 can prevent the user U from being incorrectly determined as not being tired even though the user U is tired based on the difference in stride length of the user U made according to a situation where the user U is walking. As a result, the information processing apparatus 20 can accurately determine whether the user is tired. The stride length of the user U when he or she is not tired in every situation where the user U is walking may be measured in advance from experiments, may be calculated in theoretical calculation or the like, may be estimated using a machine learning model, or may be determined in other methods.

After acquiring the user stride-length information and the walking-related information, the information processing apparatus 20 determines whether the user U is tired based on the acquired user stride-length information and the walking-related information, and the determination criterion information stored in advance. More specifically, after acquiring the user stride-length information and the walking-related information, the information processing apparatus 20 extracts the threshold information associated with the walking-related information from the determination criterion information. At this time, that a value indicated by information of a type included in the determination criterion information matches a value indicated by information of the type included in the walking-related information means, for example, that the value indicated by the information of the type included in the walking-related information is included in a predetermined range in which the value indicated by the information of the type included in the determination criterion information is set as a central value. The predetermined range is, for example, the range of the center value ±10%, but it is not limited thereto. Furthermore, that a value indicated by information of a type included in the determination criterion information matches a value indicated by information of the type included in the walking-related information may be defined using another method. Then, the information processing apparatus 20 determines whether the stride length indicated by the user stride-length information is less than the threshold indicated by the extracted threshold information. When it is determined that the stride length is equal to or greater than the threshold, the information processing apparatus 20 determines that the user U is not tired. When it is determined that the stride length is less than the threshold, the information processing apparatus 20 determines that the user U is tired. Thus, the information processing apparatus 20 can accurately determine whether the user U is tired based on how the feet of the user U move.

After determining whether the user U is tired, the information processing apparatus 20 performs processing in accordance with the result of determination of whether the user U is tired. A specific example of the processing will be described later.

Further, the threshold information described above may be replaced with information indicating a predetermined range as a range including stride lengths of the user U when he or she is not tired in a situation indicated by the walking-related information associated with the threshold information. Examples of the range including stride lengths of the user U when he or she is not tired in that situation may be a range indicated by one standard deviation of the stride lengths of the user U when he or she is not tired in that situation, or another range that can be statistically calculated for the stride lengths of the user U when he or she is not tired. Furthermore, the threshold information described above may be the average value, the median value, and the like of the stride lengths of the user U when he or she is not tired in that situation, or may be other statistical values of the stride lengths of the user U when he or she is not tired in that situation.

In addition, the information processing apparatus 20 may determine whether the user U is tired based on user pitch information instead of the user stride-length information. This is because, if a person is tired while walking, the pitch of the person tends to decrease compared to the pitch of the person when he or she is not tired. In this case, the information processing apparatus 20 calculates the pitch of the user U instead of the stride length of the user U. Here, the pitch of the user U is the number of steps per unit time of the user U. A method of calculating the pitch of the user U based on detection data may be a known method, or may be a method to be developed in the future.

In addition, the information processing apparatus 20 may determine whether the user U is tired based on the user stride-length information and user pitch information. In this case, the information processing apparatus 20 calculates the pitch of the user U and the stride length of the user U. In addition, in this case, instead of the aforementioned threshold information, the determination criterion information includes a combination of stride-length threshold information indicating a threshold of a stride length of the user U and a pitch threshold information indicating a threshold of a pitch of the user U. In addition, in this case, the information processing apparatus 20 may determine that the user U is tired, for example, if the stride length of the user U is less than the threshold indicated by the stride-length threshold information, and the pitch of the user U is less than the threshold indicated by the pitch threshold information. In addition, in this case, the information processing apparatus 20 may determine that the user U is tired, for example, if the stride length of the user U is less than the threshold indicated by the stride-length threshold information, or if the pitch of the user U is less than the threshold indicated by the pitch threshold information.

Furthermore, the information processing apparatus 20 may not acquire walking-related information. In this case, the determination criterion information may be a threshold information indicating a certain predetermined threshold, or may be information including the threshold information.

In addition, the information processing apparatus 20 may input detection data received from the electronic device 10 into a machine learning model that has learned the determination criterion information and cause the model to determine whether the user U is tired. Here, the model may be any machine learning model as long as it can learn the determination criterion information, and it is a model of a type in which it can be determined whether the user U is tired based on the detection data.

The mobile terminal 30 transmits various requests to the information processing apparatus 20, and receives various types of data from the information processing apparatus 20 as responses to the requests. In addition, the mobile terminal 30 transmits various requests to the electronic device 10 to control the electronic device 10. Furthermore, upon receiving detection data from the electronic device 10, the mobile terminal 30 transmits the received detection data to the information processing apparatus 20. In other words, the mobile terminal 30 relays transmission and reception of detection data between the electronic device 10 and the information processing apparatus 20.

The mobile terminal 30 is an information processing terminal that can be carried by the user U in this example, and is a tablet PC, a personal digital assistant (PDA), a multi-function mobile telephone terminal (smartphone), a smart watch, or a head-mounted display, for example, but it is not limited thereto. Further, the mobile terminal 30 may be a portable information processing terminal that the user U lends from another person, or may be another information processing terminal.

The mobile terminal 30 performs transmission and/or reception of various kinds of data with respect to the information processing apparatus 20 in wireless communication based on a predetermined second standard. The second standard may be, for example, the standard for Long Term Evolution (LTE) or the like, the standard for Wi-Fi (trade name) or the like, or another standard for wireless communication.

### Hardware Configuration of Electronic Device

A hardware configuration of the electronic device 10 will be described below with reference to FIG. 2. FIG. 2 is a diagram illustrating an example of a hardware configuration of the electronic device 10.

The electronic device 10 includes, for example, a first processor 11, a first storage unit 12, a first communication unit 13, and a detection unit 14. These constituent components are communicatively coupled to each other via a bus. The electronic device 10 also communicates with the mobile terminal 30 via the first communication unit 13.

The first processor 11 is, for example, a central processing unit (CPU). Further, the first processor 11 may be another processor such as a field programmable gate array (FPGA), instead of a CPU. The first processor 11 executes various programs stored in the first storage unit 12.

The first storage unit 12 is a storage apparatus including, for example, a solid-state drive (SSD), an electronically erasable programmable read only memory (EEPROM), a read only memory (ROM), or a random-access memory (RAM). Further, the first storage unit 12 may be an externally mounted storage apparatus coupled by a digital input/output port such as a Universal Serial Bus (USB) or the like instead of those built into the electronic device 10. The first storage unit 12 stores various kinds of information to be processed by the electronic device 10 and various programs.

The first communication unit 13 is, for example, a communication apparatus including an antenna for wireless communication.

The detection unit 14 includes a first detector 141, a second detector 142, and a third detector 143.

The first detector 141 is an acceleration sensor that detects an acceleration.

The second detector 142 is an angular velocity sensor that detects an angular velocity. In the following, a case in which the second detector 142 is a gyro sensor will be described as an example. Further, the second detector 142 may be another sensor that detects an angular velocity, instead of a gyro sensor.

The third detector 143 is a position data receiver that receives position data indicating a position. In the following, a case in which the third detector 143 is a GPS receiver will be described as an example. Further, the third detector 143 may be another receiver capable of receiving data indicating a position measured by a GNSS, instead of the GPS receiver, as position data.

### Hardware Configuration of Information Processing Apparatus

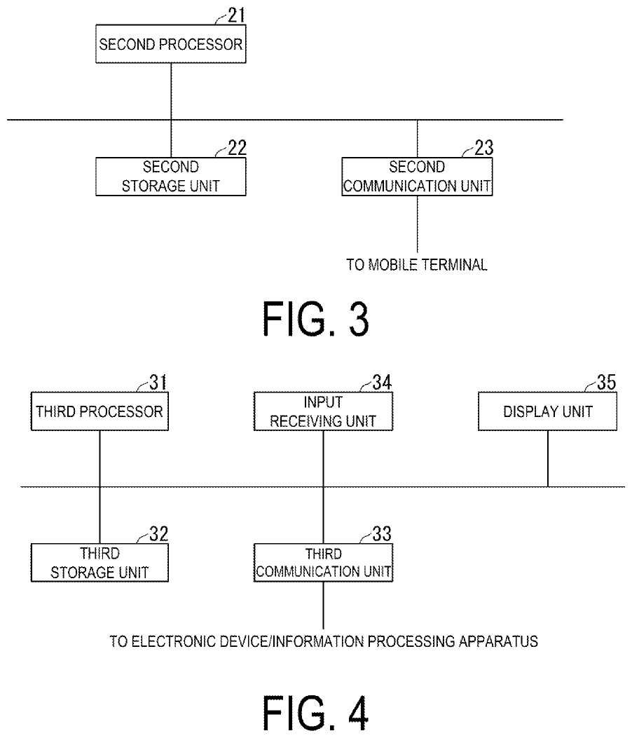

Next, a hardware configuration of the information processing apparatus 20 will be described with reference to FIG. 3. FIG. 3 is a diagram illustrating an example of a hardware configuration of the information processing apparatus 20.

The information processing apparatus 20 includes, for example, a second processor 21, a second storage unit 22, and a second communication unit 23. These constituent components are communicatively coupled to each other via a bus. The information processing apparatus 20 also communicates with the mobile terminal 30 via the second communication unit 23.

The second processor 21 is, for example, a CPU. Further, the second processor 21 may be another processor such as an FPGA, instead of a CPU. The second processor 21 executes various programs stored in the second storage unit 22.

The second storage unit 22 is a storage apparatus including, for example, a hard disk drive (HDD), an SSD, an EEPROM, a ROM, or a RAM. Further, the second storage unit 22 may be an externally mounted storage apparatus coupled by a digital input/output port such as a USB, instead of those built into the information processing apparatus 20. The second storage unit 22 stores various kinds of information, various images, and various programs to be processed by the information processing apparatus 20. For example, the second storage unit 22 stores the aforementioned determination criterion information.

The second communication unit 23 is, for example, a communication apparatus including an antenna for wireless communication.

### Hardware Configuration of Mobile Terminal

A hardware configuration of the mobile terminal 30 will be described below with reference to FIG. 4. FIG. 4 is a diagram illustrating an example of a hardware configuration of the mobile terminal 30.

The mobile terminal 30 includes, for example, a third processor 31, a third storage unit 32, a third communication unit 33, an input receiving unit 34, and a display unit 35. These constituent components are communicatively coupled to each other via a bus. The mobile terminal 30 also communicates with each of the electronic device 10 and the information processing apparatus 20 via the third communication unit 33.

The third processor 31 is, for example, a CPU. Further, the third processor 31 may be another processor such as an FPGA, instead of a CPU. The third processor 31 executes various programs stored in the third storage unit 32.

The third storage unit 32 is a storage apparatus including, for example, an SSD, an EEPROM, a ROM, or a RAM. Further, the third storage unit 32 may be an externally mounted storage apparatus coupled by a digital input/output port such as a USB, instead of those built into the mobile terminal 30. The third storage unit 32 stores various kinds of information, various images, and various programs to be processed by the mobile terminal 30.

The third communication unit 33 is, for example, a communication apparatus including an antenna for wireless communication.

The input receiving unit 34 is, for example, an input apparatus including a hard key or a touch pad. The input receiving unit 34 may be integrated with the display unit 35 as a touch panel.

The display unit 35 is, for example, a display apparatus including a display.

### Functional Configuration of Electronic Device and Information Processing Apparatus

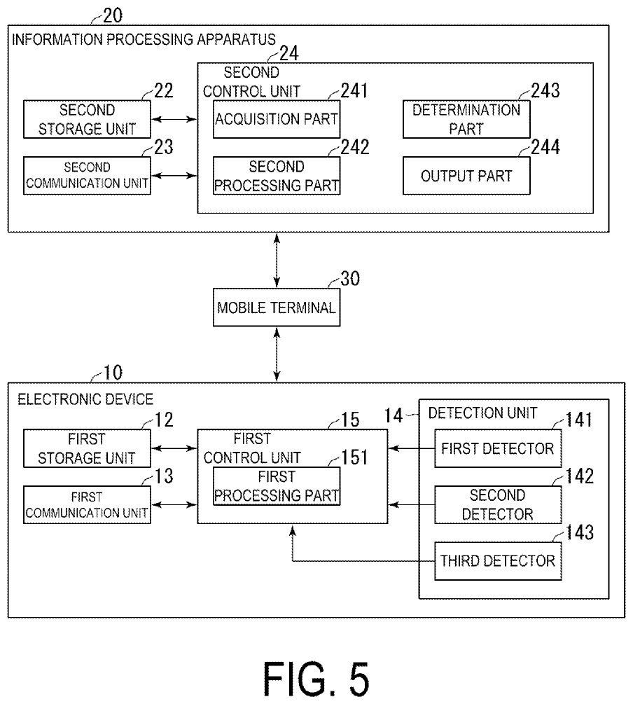

A functional configuration of each of the electronic device 10 and the information processing apparatus 20 will be described below with reference to FIG. 5. FIG. 5 is a diagram illustrating an example of a functional configuration of each of the electronic device 10 and the information processing apparatus 20.

The electronic device 10 includes, for example, the first storage unit 12, the first communication unit 13, the detection unit 14, and a first control unit 15.

The first control unit 15 controls the entire electronic device 10. The first control unit 15 includes, for example, a first processing part 151. Such a functional part included in the first control unit 15 is achieved, for example, by the first processor 11 executing various programs stored in the first storage unit 12. In addition, some or all of the functional parts may be hardware functional parts such as large-scale integration (LSI) and an application specific integrated circuit (ASIC). Further, the first control unit 15 may include other functional parts in addition to the first processing part 151.

The first processing part 151 performs various processing operations in accordance with requests received from the mobile terminal 30.

The information processing apparatus 20 includes, for example, the second storage unit 22, the second communication unit 23, and a second control unit 24.

The second control unit 24 controls the entire information processing apparatus 20. The second control unit 24 includes, for example, an acquisition part 241, a second processing part 242, a determination part 243, and an output part 244. The functional parts included in the second control unit 24 is achieved, for example, when the second processor 21 executes various programs stored in the second storage unit 22. Furthermore, some or all of the functional parts may be hardware functional parts such as an LSI or an ASIC. Further, the second control unit 24 may include other functional parts in addition to the acquisition part 241, the second processing part 242, the determination part 243, and the output part 244.

The acquisition part 241 acquires various types of data received by the information processing apparatus 20 from the electronic device 10. The acquisition part 241 acquires, for example, detection data received by the information processing apparatus 20 from the electronic device 10.

The second processing part 242 performs various processing operations according to operations received from the user U via the mobile terminal 30. For example, the second processing part 242 acquires the user stride-length information based on the detection data acquired by the acquisition part 241.

The determination part 243 performs various kinds of determination performed by the information processing apparatus 20. For example, the determination part 243 determines whether the user U is tired based on the user stride-length information acquired by the second processing part 242.

The output part 244 outputs various kinds of information according to operations received from the user U via the mobile terminal 30. For example, the output part 244 outputs information indicating the result determined by the determination part 243 to another apparatus such as the mobile terminal 30.

### Processing of Information Processing Apparatus to Determine Whether User is Tired

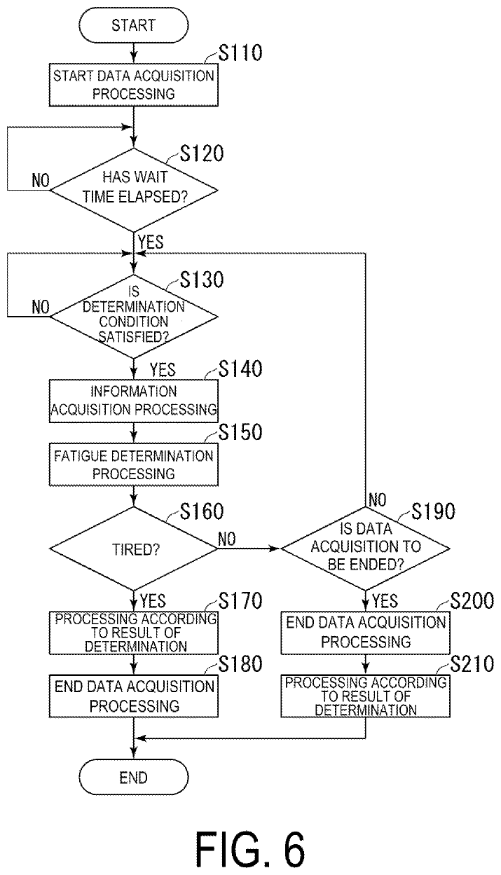

Processing of the information processing apparatus 20 to determine whether user is tired will be described below with reference to FIG. 6. FIG. 6 is a diagram illustrating an example of a process flow for the information processing apparatus 20 to determine whether a user U is tired. In the following, a case in which the information processing apparatus 20 receives, from the user via the mobile terminal 30, an operation of starting acquisition of detection data at a timing before the processing of step 5110 illustrated in FIG. 6 is performed will be described as an example. Furthermore, in the following, a case in which the user U is wearing the left foot shoe SL with the left-foot electronic device 10L attached thereto on his or her left foot and wearing the right foot shoe SR with the right-foot electronic device 10R attached thereto on his or her right foot as illustrated in FIG. 1 at the aforementioned timing will be described as an example. Furthermore, in the following, a case in which the user U starts walking at that timing will be described as an example. Furthermore, in the following, a case in which the above-described determination criterion information is stored in the second storage unit 22 at that timing will be described as an example.

After receiving the operation of starting the acquisition of the detection data, the acquisition part 241 starts data acquisition processing via the mobile terminal 30 (step S110). Here, the data acquisition processing is processing of the acquisition part 241 to acquire the detection data from the electronic device 10 each time a predetermined sampling period elapses.

Next, the second processing part 242 waits until a predetermined wait time elapses (step S120). Here, the predetermined wait time is the time required until a sufficient number of pieces of detection data for accurately executing the fatigue determination processing in step S140, which will be described later, from the timing at which the processing of step S110 is performed, and although the time is about five minutes, it may be shorter than five minutes, or longer than five minutes. The predetermined waiting time may be determined through a prior experiment, theoretical calculation, or the like, or may be determined using other methods. Further, the information processing apparatus 20 may set a time desired by the user U as the predetermined wait time according to an operation of the user U via the mobile terminal 30. Furthermore, in the processing of the flowchart illustrated in FIG. 6, the processing of step S120 may be omitted.

When the second processing part 242 determines that the predetermined wait time has elapsed (YES in step S120), the second processing part 242 waits until a predetermined determination condition is satisfied (step S130). Here, the predetermined determination condition is a condition that triggers periodic determination of whether the user U is tired. The predetermined determination condition is that, for example, a predetermined determination interval has passed from the timing at which the second processing part 242 determined that the predetermined wait time had elapsed in step S120 or the timing at which the determination condition was determined to be satisfied in step S130 performed in the previous processing round. Further, the predetermined determination condition may be another condition that triggers periodic determination of whether the user U is tired. Furthermore, although the predetermined determination interval is, for example, one minute, it may be shorter than one minute, or longer than one minute.

When it is determined that the predetermined determination condition has been satisfied (YES in step S130), the second processing part 242 acquires the above-described walking-related information along with user stride-length information based on all detection data acquired by the acquisition part 241 in the period from the timing at which step S110 was performed to the current time (step S140). In FIG. 6, the processing of step S140 is indicated by “information acquisition processing”. Further, since the method of acquiring user stride-length information based on the corresponding detection data has already been described, description thereof is omitted here. Furthermore, since the method of acquiring walking-related information based on the corresponding detection data has already been described, description thereof is omitted here.

Next, the determination part 243 performs fatigue determination processing based on the user stride-length information acquired by the second processing part 242 in step S140 (step S150). Here, the fatigue determination processing is processing in which first processing of the determination part 243 reading the determination criterion information stored in advance in the second storage unit 22 from the second storage unit 22 and second processing of determining whether the user U is tired based on the determination criterion information read in the first processing and the user stride-length information are performed in order. Further, since the method of the information processing apparatus 20 for determining whether the user U is tired based on the corresponding determination criterion information and the corresponding user stride-length information in the second processing has already been described, description thereof is omitted here. In addition, the determination part 243 may perform the first processing and the second processing in parallel.

Next, the second processing part 242 determines whether the user U has been determined as being tired in the fatigue determination processing in step S150 (step S160). In FIG. 6, the processing of step S160 is indicated by “tired?”.

When the second processing part 242 determines that the user U has been determined as being tired in the fatigue determination processing of step S150 (YES in step S160), the second processing part 242 performs processing according to the fact that the user U is tired (S170). In FIG. 6, the processing of step S170 is indicated by “processing according to determination result”. The processing of step S170 is an example of processing according to the result of determining whether the user is tired. Here, the processing of step S170 will be described.

In step S170, the second processing part 242 specifies the menu for meals containing the nutrients that help alleviate fatigue of the user U, and outputs the meal menu information indicating the specified menu to the mobile terminal 30 via the second communication unit 23, for example. Thus, the information processing apparatus 20 can cause the display of the mobile terminal 30 to display the meal menu information. Here, a method of specifying the menu for meals containing nutrients that help alleviate fatigue of the user may be a known method, or may be a method to be developed in the future. For example, the second processing part 242 may search the Internet for a corresponding menu and randomly determine a menu to be recommended to the user U from among the one or more menus obtained as a result of the search. Furthermore, for example, the second processing part 242 may use a machine learning model to output a menu to be recommended to the user U to the model. Further, the second processing part 242 may specify a menu to be recommended to the user U using other methods.

Here, for example, JP-A-2020-181313 describes an apparatus that suggests a meal that help alleviate the actual feeling of fatigue of a subject, which is measured by a sensor attached to a bedding of the subject such as a mattress based on the level of fatigue input to an electronic device by the subject. However, such an apparatus has difficulty suggesting a meal suitable for alleviating the level of fatigue of the subject during the day. The apparatus utilizes the sensor attached to the bedding in order to suggest a meal that help alleviate the level of fatigue of the subject. Thus, the apparatus is not able to suggest a meal suitable for alleviating the level of fatigue of the subject during the day when the subject is away from the bedding. In contrast, the information processing system 1 outputs meal menu information as the processing of step S170 to determine whether the user U is tired in all time slots in which the user U lives wearing the first shoes S, and when it is determined that the user U is tired, the meal menu information indicating a menu for meals containing nutrients that help alleviate the fatigue of the user U can be output. As a result, the information processing system 1 can suggest of a meal suitable for alleviating the fatigue of the user U in all the time slots in which the user U lives wearing the first shoes S.

In addition, in step S170, the second processing part 242 may output, for example, information indicating that the user U is tired to a server or the like that manages attendance of the user U at the company, instead of the configuration in which the meal menu information is output to the mobile terminal 30. Thus, the information processing apparatus 20 can help the company of the user U to precisely ascertain, for example, the degree of contribution of the user U to the company. As a result, this makes it possible for the company to precisely evaluate the employees, for example.

Furthermore, in step S170, the second processing part 242 may output, for example, information indicating that the user U is tired to an information processing apparatus with a function of playing music, instead of having the configuration in which the meal menu information is output to the mobile terminal 30. Examples of the information processing apparatus include the mobile terminal 30, and an information processing apparatus of the company of the user U. Thus, for example, the information processing apparatus 20 can cause music for a refreshing exercise to be played, or the like at the company of the user U according to the fatigue of the employees. As a result, the information processing apparatus 20 can improve the living environment of the user U, for example.

Furthermore, in step S170, the second processing part 242 may output, for example, action plan information indicating an action plan for alleviating fatigue of the user U to the mobile terminal 30 via the second communication unit 23, instead of having the configuration in which the meal menu information is output to the mobile terminal 30. Thus, the information processing apparatus 20 can cause the display of the mobile terminal 30 to display the action plan information. Here, although the action plan information is, for example, information indicating a suggestion for a travel to a recommended hot spring inn, or information indicating a suggestion for a trip to a recommended tourists' attraction, it is not limited thereto. Such action plan information may be output, for example, on a holiday, on the eve of a holiday, or the like, by the information processing apparatus 20 based on the result determined by the information processing apparatus 20 on a weekday. A method of specifying a recommended action plan may be a known method, or may be a method to be developed in the future. For example, the second processing part 242 may search the Internet for an action plan and randomly determine an action plan to be recommended to the user U from among the one or more action plans obtained as a result of the search. Furthermore, for example, the second processing part 242 may use a machine learning model to output the action plan to be recommended to the user U to the model. Further, the second processing part 242 may specify an action plane to be recommended to the user U using other methods.

After the processing of step S170 is performed, the acquisition part 241 ends the data acquisition processing via the mobile terminal 30 (step S180), and ends the processing of the flowchart illustrated in FIG. 6.

Meanwhile, when it is determined that the user U is not determined as being tired in the fatigue determination processing of step S150 (NO in step S160), the second processing part 242 determines whether the acquisition of the detection data is to be ended (step S190). For example, in step S190, the second processing part 242 determines that the acquisition of the detection data is to be ended when the information processing apparatus 20 receives the operation to end acquisition of the detection data from the user via the mobile terminal 30. In addition, for example, the second processing part 242 determines that the acquisition of the detection data is not to be ended when the information processing apparatus 20 receives the operation from the user via the mobile terminal 30 in step S190.

When it is determined that the acquisition of the detection data is not to be ended (NO in step S190), the second processing part 242 proceeds to step S130, and waits again until the predetermined determination condition is satisfied.

On the other hand, when the second processing part 242 determines that the acquisition of the detection data is to be ended (YES in step S190), the acquisition part 241 ends the data acquisition processing via the mobile terminal 30 (step S200).

Next, the second processing part 242 performs processing according to the fact that the user U is not tired (step S210). In FIG. 6, the processing of step S210 is indicated by “processing according to determination result”. The processing of step S210 is an example of processing according to the result of determining whether the user is tired. Here, the processing of step S210 will be described.

In step S210, the second processing part 242 specifies the menu for the user U's favorite meals and outputs meal menu information indicating the specified menu to the mobile terminal 30 via the second communication unit 23, for example. Thus, the information processing apparatus 20 can cause the display of the mobile terminal 30 to display the meal menu information. Here, a method of specifying the menu for the user's favorite meals may be a known method, or may be a method to be developed in the future. For example, the second processing part 242 may search the Internet for the history of use of social network services (SNSs) of the user U and randomly determine a menu to be recommended to the user U from among the one or more menus obtained as a result of the search. Furthermore, for example, the second processing part 242 may use a machine learning model to output a menu to be recommended to the user U to the model. Further, the second processing part 242 may specify a menu to be recommended to the user U using other methods.

In addition, in step S210, the second processing part 242 may output, for example, information indicating that the user U is not tired to a server or the like that manages attendance of the user U at the company, instead of having the configuration in which the meal menu information is output to the mobile terminal 30. Thus, the information processing apparatus 20 can help the company of the user U to precisely ascertain, for example, the degree of contribution of the user U to the company. As a result, this makes it possible for the company to precisely evaluate the employees, for example.

Furthermore, in step S210, the second processing part 242 may output, for example, information indicating that the user U is not tired to an information processing apparatus with a function of playing music, instead of having the configuration in which the meal menu information is output to the mobile terminal 30. Examples of the information processing apparatus include the mobile terminal 30, and an information processing apparatus of the company of the user U. Thus, for example, the information processing apparatus 20 can cause music that help the user concentrate to be played, or the like at the company of the user U. As a result, the information processing apparatus 20 can improve the living environment of the user U, for example.

Furthermore, in step S210, the second processing part 242 may output, for example, action plan information indicating an action plan after work of the user U to the mobile terminal 30 via the second communication unit 23, instead of having the configuration in which the meal menu information is output to the mobile terminal 30. Thus, the information processing apparatus 20 can cause the display of the mobile terminal 30 to display the action plan information. Here, although the action plan information is, for example, information indicating a suggestion for the hobby of the user U, or information indicating a suggestion for a visit to a recommended restaurant, it is not limited thereto. A method of specifying a recommended action plan may be a known method, or may be a method to be developed in the future. For example, the second processing part 242 may search the Internet for the history of use of the SNSs of the user U and randomly determine an action plan to be recommended to the user U from among the one or more action plans obtained as a result of the search. Furthermore, for example, the second processing part 242 may use a machine learning model to output the action plan to be recommended to the user U to the model. Further, the second processing part 242 may specify an action plane to be recommended to the user U using other methods.

After the processing of step S210 is performed, the second processing part 242 ends the processing of the flowchart illustrated in FIG. 6.

As described above, the information processing apparatus 20 acquires, based on the detection data acquired by the electronic device 10 attached to the first shoes S, user stride-length information indicating the stride length of the user U wearing the first shoes S, and determines whether the user U is tired based on the acquired user stride-length information. Thus, the information processing apparatus 20 can accurately determine whether the user U is tired based on the stride length of the user U, that is, how the feet of the user U move.

Here, the information processing apparatus 20 may determine whether the user U is physically tired and whether the user U is mentally tired based on the user stride-length information and the walking-related information in the fatigue determination processing of step S150. In this case, if the user U is determined as being tired in the time slot in which the user U goes to work, for example, the information processing apparatus 20 determines that the user U is mentally tired since the user U is highly likely not yet to lose his or her physical strength. On the other hand, if the user U is determined as being tired in the time slot in which the user U leaves work, for example, the information processing apparatus 20 determines that the user U is physically tired since the user U is highly likely to have already lost his or her physical strength. Here, when such a determination is made, each of the information indicating the time slot in which the user U goes to work and the information indicating the time slot in which the user U leaves work, for example, is registered to the information processing apparatus 20 via the mobile terminal 30 by the user U. Further, when the fatigue determination processing of step S150 is performed using a machine learning model, the information processing apparatus 20 causes the machine learning model to learn the lifestyle of the user U. As a result, the information processing apparatus 20 can determine whether the user U is physically tired and whether the user U is mentally tired with higher accuracy. Furthermore, the information processing apparatus 20 may determine whether the user U is physically tired based on the speed indicated by the user speed information included in the walking-related information. In this case, the information processing apparatus 20 can determine that the user U gets physically tired as the speed becomes lower.

In addition, when whether the user U is mentally tired and whether the user U is physically tired are each determined, the information processing apparatus 20 may output meal menu information indicating a menu for a meal in accordance with a combination of the results of the two determinations in step S170. In this case, the information processing apparatus 20 outputs meal menu information indicating a different menu for each combination. For example, when it is determined that the user U is not mentally tired and that the user U is physically tired, the information processing apparatus 20 outputs meal menu information indicating a menu for a meal containing pork that is nutritionally balanced and rich in vitamins, or the like, to the mobile terminal 30 and the like. In addition, for example, when it is determined that the user U is mentally tired and that the user U is not physically tired, the information processing apparatus 20 outputs meal menu information indicating a menu for a meal made of sesame and soybean products containing tryptophan, or the like, to the mobile terminal 30 and the like. Here, tryptophan is an essential amino acid contained in sesame, soy product, and the like, which is a material of the neurotransmitter “serotonin” that calms emotions and reduces stress. In other words, the information processing apparatus 20 can encourage the user U to consume tryptophan to alleviate mental fatigue.

Furthermore, the information processing apparatus 20 may calculate the value indicating the level of fatigue of the user U in the fatigue determination processing in step S150. For example, the information processing apparatus 20 may calculate a discrete numerical value associated with the magnitude of the difference between every stride length indicated by the user stride-length information and a threshold indicated by threshold information associated with the walking-related information as a value indicating the degree of fatigue of the user U. In this case, as the magnitude of the difference is greater, a value indicating that the user U is more seriously tired is associated with the magnitude of the difference. In addition, the discrete numerical value indicates that the user U is more seriously tired as the value becomes smaller or the value becomes greater. When such a value indicating the degree of fatigue of the user U is calculated as described above, the information processing apparatus 20 may output information indicating the value to another apparatus to be displayed in step S170 and step S210.

### Processing of Information Processing Apparatus to Acquire Detection Information Used to Generate Determination Criterion Information

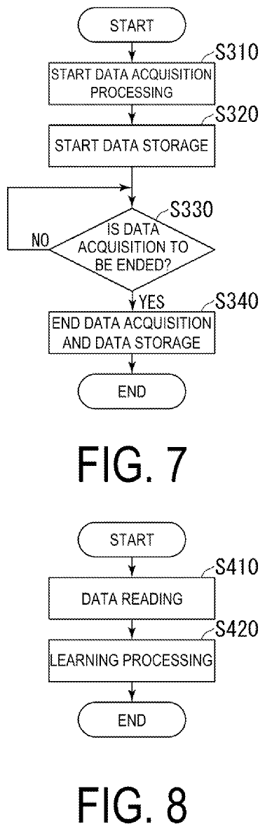

Here, the information processing apparatus 20 may generate determination criterion information as described above. In this case, the information processing apparatus 20 acquires detection data used to generate the determination criterion information, and generates the determination criterion information based on the acquired detection data. Thus, processing of the information processing apparatus 20 to acquire detection data used to generate determination criterion information will be described below referring to FIG. 7. FIG. 7 is a diagram illustrating an example of a process flow in which the information processing apparatus 20 acquires detection data used to generate determination criterion information. In the following, a case in which the information processing apparatus 20 receives, from the user via the mobile terminal 30, an operation of starting acquisition of detection data used to generate determination criterion information at a timing before the processing of step S310 illustrated in FIG. 7 is performed will be described as an example. Furthermore, in the following, a case in which the user U is wearing the left foot shoe SL with the left-foot electronic device 10L attached thereto on his or her left foot and wearing the right foot shoe SR with the right-foot electronic device 10R attached thereto on his or her right foot as illustrated in FIG. 1 at the aforementioned timing will be described as an example. Furthermore, in the following, a case in which the user U starts walking at that timing will be described as an example. The information processing apparatus 20 repeats the processing of the flowchart illustrated in FIG. 7 each time the user U performs the corresponding operation. Thus, the information processing apparatus 20 can acquire the detection data in various time slots of the daily life of the user U, and as a result, the information processing apparatus 20 can generate determination criterion information including threshold information associated with various kinds of walking-related information according to the daily life of the user U.

After receiving the operation of starting the acquisition of the detection data used to generate the determination criterion information, the acquisition part 241 starts data acquisition processing via the mobile terminal 30 (step S310).

Next, the acquisition part 241 starts processing of causing the acquired detection data to be stored in the second storage unit 22 each time the detection data is acquired from the data acquisition processing started in step S310 (step S320). In FIG. 7, the processing of step S320 is indicated by “start data storage”. Note that, when the detection data is to be stored in the second storage unit 22 in step S320, for example, the acquisition part 241 causes the second storage unit 22 to store data identification information indicating data to be used to generate the determination criterion information in association with the detection data.

Next, the acquisition part 241 waits until an operation of ending the acquisition of the detection data used to generate the determination criterion information is received (step S330). In FIG. 7, the processing of step S330 is indicated by “end data acquisition?”.

When it is determined that the operation of ending the acquisition of the detection data used to generate the determination criterion information has been received (YES in step S330), the acquisition part 241 ends the two processing operations that are the data acquisition processing started in step S310 and the processing started in step S320 (step S340), and ends the processing of the flowchart illustrated in FIG. 7. In FIG. 7, the processing of step 5340 is indicated by “end data acquisition and data storage”.

### Processing of Information Processing Apparatus to Generate Determination Criterion Information

Processing in which the information processing apparatus 20 generates the determination criterion information based on the detection data acquired in the processing of the flowchart illustrated in FIG. 7 will be described with reference to FIG. 8. FIG. 8 is a diagram illustrating an example of a process flow in which the information processing apparatus 20 generates the determination criterion information based on the detection data acquired in the processing of the flowchart illustrated in FIG. 7. In the following, a case in which the determination criterion information is a weight between nodes in a machine learning model will be described as an example. In addition, in the following, a case in which the information processing apparatus 20 receives, from the user via the mobile terminal 30, an operation of starting generation of determination criterion information at a timing before the processing of step S410 illustrated in FIG. 8 is performed will be described as an example. Furthermore, in the following, a case in which detection data used to generate determination criterion information is stored in the second storage unit 22 in the processing of the flowchart illustrated in FIG. 7 at that timing will be described as an example. Further, the processing of the flowchart illustrated in FIG. 8 may be performed in parallel with the processing of the flowchart illustrated in FIG. 7. In this case, the information processing apparatus 20 generates and updates the determination criterion information while acquiring the detection data.

The second processing part 242 reads the detection data associated with the above-described data identification information from the second storage unit 22 (step S410).

Next, the second processing part 242 acquires the user stride-length information and the walking-related information based on the detection data read from the second storage unit 22 in step S410. The second processing part 242 inputs the acquired user stride-length information and the walking-related information into the machine learning model and causes the model to learn the information (step S420), and ends the processing of the flowchart illustrated in FIG. 8. In FIG. 8, the processing of step S420 is indicated by “learning processing”. Here, since the method for acquiring the user stride-length information and the walking-related information based on the corresponding detection data has already been described, description thereof is omitted here. In this manner, in step S420, in order to cause the machine learning model to learn combinations of the user stride-length information and the walking-related information based on the detection data, the information processing apparatus 20 can generate a machine learning model available as determination criterion information including threshold information indicating a threshold corresponding to each situation in which the user U is walking. Further, a detailed learning method for the machine learning model may be a known method, or may be a method to be developed in the future.

Further, the information processing apparatus 20 described above may use various functions, various classes, and the like instead of the machine learning model.

Furthermore, the information processing apparatus 20 described above may acquire, based on the detection data acquired by the electronic device 10 attached to the first shoes S, the user stride-length information of the user wearing the first shoes S and determine whether the user U is injured based on the acquired user stride-length information. This is because, for example, stride lengths of the user U become shorter when at least one of the left foot and the right foot of the user U is injured. Thus, the information processing apparatus 20 can accurately determine whether the user U is injured based on how the feet of the user U move. Further, the injury of the user U may be, for example, a physical damage of the user U or an illness of the user U.

Furthermore, in the information processing system 1 described above, all of the matters described above may be combined in any manner.

As described above, an information processing apparatus according to the embodiment acquires information indicating how the feet of a user wearing shoes move based on data acquired by an electronic device attached to the shoes and determines whether the user is tired based on the acquired information. Thus, the information processing apparatus can accurately determine whether the user is tired based on how the feet of the user move. Here, in the example described above, the information processing apparatus 20 is an example of the aforementioned information processing apparatus. In addition, in the example described above, the first shoes S are an example of the aforementioned shoes. In addition, in the example described above, the electronic device 10 is an example of the aforementioned electronic device. In addition, in the example described above, each of the acceleration data, angular velocity data, and position data is an example of the aforementioned data. In addition, in the example described above, the user U is an example of the aforementioned user. Here, in the example described above, each of the user stride-length information and the user pitch information is an example of the aforementioned information.

Furthermore, the information processing apparatus may employ a configuration in which the information indicating how the feet of the user move includes user stride-length information indicating a stride length of the user.

In addition, the information processing apparatus may employ a configuration in which the information indicating how the feet of the user move includes user pitch information indicating a pitch of the feet of the user.

In addition, the information processing apparatus may employ a configuration in which whether the user is tired is determined based on a predetermined threshold.

In addition, the information processing apparatus may employ a configuration in which whether the user is tired is determined based on the acquired information and a threshold predetermined for each time slot.

In addition, the information processing apparatus may employ a configuration in which whether the user is tired is determined based on the acquired information and a threshold predetermined for each walking speed of the user.

In addition, the information processing apparatus may employ a configuration in which whether the user is tired is determined based on the acquired information and a threshold predetermined for each slope of the road surface.

In addition, the information processing apparatus may use a configuration for determining whether the user is physically tired and the user is mentally tired based on the acquired information.

In addition, the information processing apparatus may employ a configuration in which meal menu information indicating a menu for a meal in accordance with the result of determination is output after whether the user is tired is determined.

In addition, the information processing apparatus may employ a configuration in which, after whether the user is physically tired and whether the user is mentally tired are each determined, meal menu information indicating a menu for a meal in accordance with a combination of the results of the determination is output.

In addition, the information processing apparatus may employ a configuration in which meal menu information indicating a different menu for each combination is output.

In addition, the information processing apparatus may employ a configuration in which the data acquired by the electronic device includes at least one of acceleration data indicating an acceleration of the first shoes or angular velocity data indicating an angular velocity of the first shoes.

Furthermore, the information processing apparatus may employ a configuration in which the data acquired by the electronic device includes position data indicating a position of the user.

In addition, the information processing apparatus acquires information indicating how the feet of the user wearing the shoes move based on the data acquired by the electronic device attached to the shoes and determines whether the user is injured based on the acquired information. Thus, the information processing apparatus can accurately determine whether the user is injured based on how the feet of the user move.

Although the embodiments of this disclosure have been described in detail with reference to the drawings, the specific configurations are not limited to these embodiments, and may be modified, substituted, deleted, and the like without departing from the spirit of this disclosure.

In addition, a program for achieving the functions of any constituent units of the apparatus described above may be recorded in a computer-readable recording medium, and the program may be read and executed by a computer system. Here, the apparatus is, for example, the electronic device 10, the information processing apparatus 20, or the mobile terminal 30. Further, the “computer system” mentioned here is assumed to include hardware such as an operating system (OS) or a peripheral apparatus. Furthermore, the “computer-readable recording medium” refers to a portable medium such as a flexible disk, a magneto-optical disk, a ROM, and a compact disc (CD)-ROM, and a storage apparatus such as a hard disk built into a computer system. Furthermore, the “computer-readable recording medium” is assumed to include one that holds a program for a certain period of time, such as volatile memory inside a computer system serving as a server or a client when a program is transmitted via a network such as the Internet or a communication line such as a telephone line.

In addition, the program described above may be transmitted from a computer system storing the program in a storage apparatus or the like to another computer system via a transmission medium or using transmission waves in a transmission medium. Here, the “transmission medium” for transmitting a program refers to a medium having a function of transmitting information, like a network such as the Internet or a communication line such as a telephone line.

In addition, the program described above may be one to achieve some of the functions described above. Furthermore, the program described above can be a so-called differential file or a differential program that can achieve the above-described functions in combination with a program already recorded in the computer system.

What is claimed is:

1. An information processing apparatus configured to acquire information indicating how a foot of a user wearing a shoe moves based on data acquired by an electronic device attached to the shoe and determine whether the user is tired based on the acquired information.

2. The information processing apparatus according to claim 1, wherein
the information includes user stride-length information indicating a stride length of the user.

3. The information processing apparatus according to claim 1, wherein
the information includes user pitch information indicating a pitch of the foot of the user.

4. The information processing apparatus according to claim 1, wherein
whether the user is tired is determined based on the acquired information and a threshold being predetermined.

5. The information processing apparatus according to claim 4, wherein
whether the user is tired is determined based on the acquired information and the threshold predetermined for each time slot.

6. The information processing apparatus according to claim 4, wherein
whether the user is tired is determined based on the acquired information and the threshold predetermined for each walking speed of the user.

7. The information processing apparatus according to claim 4, wherein
whether the user is tired is determined based on the acquired information and the threshold predetermined for each slope of road surface.

8. The information processing apparatus according to claim 1, wherein
whether the user is physically tired and whether the user is mentally tired are each determined based on the acquired information.

9. The information processing apparatus according to claim 1, wherein
after whether the user is tired is determined, meal menu information indicating a menu for a meal in accordance with a determination result is output.

10. The information processing apparatus according to claim 8, wherein
after whether the user is physically tired and whether the user is mentally tired are each determined, meal menu information indicating a menu for a meal in accordance with a combination of determination results is output.

11. The information processing apparatus according to claim 10, wherein
the meal menu information indicating a different menu for each combination is output.

12. The information processing apparatus according to claim 1, wherein
the data includes at least one of acceleration data indicating an acceleration of the shoe or angular velocity data indicating an angular velocity of the shoe.

13. The information processing apparatus according to claim 12, wherein
the data includes position data indicating a position of the user.

14. An information processing system comprising:
the information processing apparatus according to claim 1; and
the electronic device according to claim 1.

15. An information processing method for an information processing apparatus, the information processing method comprising:
receiving data acquired by an electronic device attached to a shoe;
acquiring information indicating how a foot of a user wearing the shoe moves based on the received data; and
determining whether the user is tired based on the acquired information.

16. A non-transitory computer-readable storage medium storing a program, the program being configured to cause a computer of an information processing apparatus to:
receive data acquired by an electronic device attached to a shoe;
acquire information indicating how a foot of a user wearing the shoe moves based on the received data; and
determine whether the user is tired based on the acquired information.

17. An information processing apparatus configured to acquire information indicating how a foot of a user wearing a shoe moves based on data acquired by an electronic device attached to the shoe and determine whether the user is injured based on the acquired information.
